(19) United States
(12) Patent Application Publication
(10) Pub. No.: US 2019/0275495 A1
(43) Pub. Date: Sep. 12, 2019

(54) ACTIVATED CARBON FILTER ARTICLES AND METHODS OF MAKING AND THEIR USE

(71) Applicant: Corning Incorporated, Corning, NY (US)

(72) Inventor: Benedict Yorke Johnson, Horseheads, NY (US)

(21) Appl. No.: 16/423,889

(22) Filed: May 28, 2019

Related U.S. Application Data
(62) Division of application No. 14/703,399, filed on May 4, 2015.
(60) Provisional application No. 62/003,671, filed on May 28, 2014.

(51) Int. Cl. B01J 20/20; B01J 20/28; B01D 46/24; B01D 15/00; B01J 20/30; B01J 20/10; B01J 20/24; B01J 20/26; C02F 1/28; C07C 7/12; B01D 53/04; C04B 38/00; C04B 26/28; B01D 15/08; C04B 35/636; C04B 35/532; C04B 35/528

(52) U.S. Cl. CPC B01J 20/20; C04B 2111/0081; B01J 20/28064; B01J 20/28045; B01D 46/2418; B01D 15/00; B01J 20/3078; B01J 20/305; B01J 20/3007; B01J 20/103; B01J 20/24; B01J 20/261; C02F 1/283; C07C 7/12; B01D 53/04; B01J 20/3042; C04B 38/0006; C04B 26/285; B01D 15/08; C04B 35/6365; C04B 35/532; C04B 35/528; B01D 46/2474; B01D 46/247; B01J 20/28066; B01D 2253/102; B01D 2253/3425; B01D 2257/7027; C04B 2235/6021; B01J 20/2803

(57) ABSTRACT

A method of making an activated carbon honeycomb filter article, as defined herein, including:
extruding a batch mixture to form an extruded honeycomb body, the batch including:
   an activated carbon powder;
   a first organic binder powder;
   a rheological plasticizing liquid;
   a porous inorganic binder powder;
   an extrusion aid; and
   water by superaddition,
drying the extruded honeycomb body; and
heat treating the dried honeycomb body. Also disclosed is an honeycomb filter article, having: an activated carbon; a porous inorganic binder powder; a BET surface area of from 950 m²/g to 1600 m²/g; a cell density of from 50 to 2000 cpsi; and a density of from 0.5 to 0.8 g/cm³.

This application claims the benefit of priority under 35 U.S.C. § 119 of U.S. Provisional Application Ser. No. 62/003,671 filed on May 28, 2014 the content of which is relied upon and incorporated herein by reference in its entirety.

The entire disclosure of publications, patents, and patent documents mentioned herein are incorporated by reference.

### BACKGROUND

The disclosure relates to an activated carbon filter article and to methods of making and their use.

### SUMMARY

In embodiments, the disclosure provides activated carbon filter articles and methods for making and their use.

### BRIEF DESCRIPTION OF DRAWINGS

In embodiments of the disclosure:

FIG. 1 shows an isometric view of an exemplary activated carbon honeycomb filter article having a cell density of, for example, from about 50 to 2,000 cells per square inch (cpsi).

FIG. 2 shows an isometric view of the article of FIG. 1 having adjacent channels plugged.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments of the claimed invention.

### Definitions

“High activity” in the context of the disclosed activated carbon filter articles refers to adsorption capacity having one or more of: the high surface area, such as a surface area of from 950 to 1600 m²/g; and the type and number of reactive or interactive functional groups present in the extruded filter article. Adsorption capacity or activity can be quantified by standard methods, for example, iodine number (milligrams of iodine adsorbed by one gram of the activated carbon).

“Include,” “includes,” or like terms means encompassing but not limited to, that is, inclusive and not exclusive.

“About” modifying, for example, the quantity of an ingredient in a composition, concentrations, volumes, process temperature, process time, yields, flow rates, pressures, viscosities, and like values, and ranges thereof, or a dimension of a component, and like values, and ranges thereof, employed in describing the embodiments of the disclosure, refers to variation in the numerical quantity that can occur, for example: through typical measuring and handling procedures used for preparing materials, compositions, composites, concentrates, component parts, articles of manufacture, or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture.

“Optional” or “optionally” means that the subsequently described event or circumstance can or cannot occur, and that the description includes instances where the event or circumstance occurs and instances where it does not.

The indefinite article “a” or “an” and its corresponding definite article “the” as used herein means at least one, or one or more, unless specified otherwise.

Abbreviations, which are well known to one of ordinary skill in the art, may be used (e.g., “h” or “hrs” for hour or hours, “g” or “gm” for gram(s), “mL” for milliliters, and “rt” for room temperature, “nm” for nanometers, and like abbreviations).

Specific and preferred values disclosed for components, ingredients, additives, dimensions, conditions, times, and like aspects, and ranges thereof, are for illustration only; they do not exclude other defined values or other values within defined ranges. The composition and methods of the disclosure can include any value or any combination of the values, specific values, more specific values, and preferred values described herein, including explicit or implicit intermediate values and ranges.

“Include,” “includes,” or like terms means encompassing but not limited to, that is, inclusive and not exclusive.

Activated carbon has been historically used, for example, for removal of odor, color pigments, and various catalytic functions. However, applications of activated carbon have increased significantly in the recent years with the advancement of activated carbon process capability. Activated carbon now enjoys widespread use in the removal of impurities from fluid (i.e., liquid or gas) streams. For example, impurities in foods, fruit juices, and alcoholic beverages can be successfully filtered using activated carbon. Likewise, activated carbon is useful in the removal of gaseous species present in low concentrations in air or gas streams such as separation processes, processes for removal of organic vapors. Activated carbon has particular utility in adsorbing and purifying fluid emissions or work streams from internal combustion engines.

Conventionally, activated carbon is used in powdered or granular form. Powdered or granular activated carbon is inconvenient to use in processes where continuous work stream flows of fluids are filtered, treated, or both. This is especially true for liquids and pumps where tightly packed carbon beds can cause significant pressure drop. In applications where the bed is vibrated during use, such as in an automobile, attrition of granules results in formation of fine particles which can be trapped in the moving fluid. The flow paths in granulated beds are random and will change with time due to the formation of fines. This may result in decrease in adsorption efficiency. The pressure drops across powdered granulated beds are high for flowing systems which results in high energy costs for pumping, and like considerations. One solution to this problem is to form the activated carbon in the shape of a honeycomb. The honeycomb geometry has the advantage of high geometric surface area available for contact and low pressure drop across the bed. In some industrial processes honeycomb geometries are necessary.

Corning Incorporated has developed various methods for fabricating activated carbon honeycombs to avoid the problems associated with packed carbon beds. Of these technologies, a common method is exemplified in U.S. Pat. No. 5,510,063, to Gadkaree, et. al, “Method of making activated carbon honeycombs having varying adsorption capacities.” The method involves combining and extruding a carbon filler material (e.g., charcoal, coal, activated carbon), optionally a pore former, an extrusion aid, and a cross-linkable resin into a green body, and then curing and carbonizing the green body. After carbonization, the product is activated using CO₂ or steam (i.e. physical activation). Although these methods have been satisfactorily used in fabricating activated carbon honeycomb structures for various applications, they are encumbered with a costly high temperature heat treatment steps. In addition, the methods are plagued with the inability to uniformly activate the carbon channel. This is a significant problem when the honeycomb body has channels that are longer than the diameter of the individual channels. In these instances the gaseous oxidant is consumed prior to reaching the full length of the channels. Furthermore, liquid phenolic resin, which is a major ingredient of the batch compositions of these prior methods is expensive, has environmental issues such as emission of formaldehyde, and requires high temperature treatment (e.g., greater than 800° C.) to carbonize and activate the carbon matrix derived from it. The high temperature treatment produces a residue from burn out of the phenolic resin that can block pores and encumber surface area in the resulting honeycomb filter article. In contrast, the disclosed binder system does not produce a residue that can block pores or encumber surface area in the resulting honeycomb filter article.

In embodiments, the present disclosure provides a extrusion batch compositions for making high activity activated carbon honeycomb structures, without or free of a phenolic resin, and avoids costly high temperature heat treatment steps associated with conventional methods of making, such as carbonization and activation. The disclosed compositions can be formulated by, for example, mixing controlled amounts of powders of activated carbon, organic binder, a porous inorganic binder, a rheological plasticizing liquid prepared from a powdered organic binder, and an extrusion aid. The rheology of the fully formulated batch provides a low stick composition having a low adhesion property, which facilitates extrusion of various honeycomb geometries having fine cell densities, such as from 50 to 2,000 cpsi, from 1,600 to 2,000 cpsi, including intermediate values and ranges and uniform and smooth, external and internal surfaces.

The disclosed method of making and the resulting filter articles relies on the proper selection and the relative amounts of inorganic and organic binders, and the non-resinous plasticizing liquid binder component used in the batch formulation. The disclosed extrusion batch compositions allow for low temperature processing of the extruded bodies. The activated carbon honeycomb structures produced can be characterized by, for example: excellent structural integrity; high adsorption capacity per unit volume; convenient manufacture at low cost; and uniform high activity (i.e., high surface area) in a given volume of the carbon body. The high adsorption activity of the disclosed activated carbon and the extruded honeycomb articles obtains from at least one of: high surface area; and the presence or absence of certain functional groups on the surface of the activated carbon.

In embodiments, the present disclosure provides methods of making filter articles containing activated carbon, which methods are free of carbonization and activation steps. In embodiments, the present disclosure provides carbon filter articles having a honeycomb structure and high adsorption activity based on high surface area properties, such as from 950 to 1600 m²/g, which is conducive to adsorption.

In embodiments, the present disclosure provides extrusion batch compositions and method for making activated carbon honeycomb structures without the use of a liquid phenolic resin, and the method eliminates the need for high temperature carbonization and activation.

In embodiments, the present disclosure provides compositions that can be formulated by mixing controlled amounts of powders of: activated carbon; organic binder; porous inorganic binder; a rheological plasticizing liquid prepared from a powdered organic binder; and one or more optional extrusion aids. Combining a carbon feedstock, which is previously activated (i.e., pre-activated carbon powder), with a non-resinous binder allows the extruded carbon articles to be processed at low temperatures, such as less than about 250° C. An aspect of the disclosed method of making uses a suitable dry non-phenolic binder, dissolving the dry binder in water to form a solution having a viscosity comparable to a solution of a phenolic resin, and combining the solution with an inorganic binder to form an organic and inorganic binder system. The plasticizing liquid can also acts as a binder, and works synergistically with the solid binders (e.g., either or both the organic and the inorganic binder) to impart significant binding strength to the activated carbon particles after post extrusion treatments. The absence of a phenolic resin and the relatively low decomposition temperatures of the organic binders used in the disclosed batch formulations allow the extruded bodies to be processed at lower temperatures, for example, less than about 250° C.

In embodiments, the disclosed method of making the carbon compositions can comprises the following general steps:

batching and extruding the batch composition at ambient temperature, e.g., from 15 to 30° C., to form an extruded body;

drying the extruded body in an air-vented oven at, e.g., about 120 to 160° C.; and

heat treating the dried body at, e.g., 200 to 250° C. in nitrogen to stabilize the honeycomb structure.

In embodiments, the products obtained from the disclosed compositions and methods provide high adsorption activity, activated carbon honeycomb structures that can be used for a variety of adsorption or filter articles and processes including, for example, water and air purification, and volatile hydrocarbon gas storage. Despite the absence of a traditional high temperature treatment step, the disclosed honeycombs possess high adsorption activity, high strength, and high structural integrity.

The present disclosure is advantaged is several aspects, including for example:

manufacturability: the disclosed extrusion method of making produces honeycomb filter articles having a high honeycomb cell density, such as from 1,600 to 2,000 cells or channel openings per square inch (cpsi).

simplicity and low processing temperature: by using the disclosed compositions, the number of steps called for in the method of making the activated honeycomb structures can be reduced by eliminating the carbonization step and the activation step that have been used in traditional methods of making; the disclosed method of making can also be accomplished free of a liquid phenolic resin, which resin absence permits the extruded bodies to be processed at relatively low temperatures (e.g., less than or equal to 250° C.).

uniform activity and scalability: the disclosed compositions and methods eliminate a significant issue associated with physical activation of phenolic resin-based carbon honeycombs, which issue is the inability to uniformly activate along and across the honeycomb channels (i.e., non-uniform activation). The non-uniform activation issue was avoided in the present disclosure by uniformly distributing activated carbon particles within the walls and on the surfaces of the honeycomb structure, which carbon particle distribution results in uniform activity throughout the activated carbon honeycomb body. Accordingly, it becomes much less problematic to scale up the process for commercial production.

cost: compared to fabrication of phenolic resin-based activated honeycombs, there are two significant cost advantages of disclosed method and compositions. The first cost advantage is the elimination of costly high temperature heat treatment steps (i.e., carbonization and activation) resulting in labor and energy savings. The second cost advantage is the use of low-cost liquid binder in place of the more costly liquid phenolic resin resulting in energy and material cost savings. These provide a significant operational cost benefits in the production of the disclosed activated carbon honeycombs. The cost advantages can provide, for example, about a 40% reduction in operational production costs of the disclosed activated carbon honeycombs compared to a phenolic resin-based process.

broad applicability: The adsorption properties of the activated carbon according of the disclosure permits its use in many applications, for example, adsorbed natural gas storage, fractionation of hydrocarbons, purification of industrial gases, anti-pollution devices, liquid-phase purification processes in food and chemical industries, water treatment, liquid-phase recovery and separation, catalysts or catalyst support, and like applications.

In embodiments, the disclosure provides a method of making an activated carbon honeycomb filter article, comprising:

extruding a batch mixture to form an extruded honeycomb body, for example, at ambient temperature of, for example, about 15 to 30° C., comprising:
an activated carbon powder in from 40 to 60 wt %;
a first organic binder powder in from 5 to 10 wt %;
a rheological plasticizing liquid in from 20 to 30 wt %, prepared from a second organic binder powder in from 5 to 10 wt % in water;
a porous inorganic binder powder in from 4 to 15 wt %;
an extrusion aid in from 1 to 3 wt %; and
water in from 50 to 100 wt % by superaddition,
   the wt % is based on the total weight of the batch ingredients excluding water added based on superaddition;

drying the extruded honeycomb body at, for example, about 120 to 160° C. for 1 to 2 hrs; and

heat treating the dried honeycomb body at, for example, 200 to 250° C. in nitrogen for 2 to 4 hrs, to stabilize the honeycomb structure and produce the activated carbon honeycomb filter article.

In embodiments, the heat treated honeycomb article can have a BET surface area of from 950 m²/g to 1600 m²/g.

In embodiments, the heat treated honeycomb filter article can have a cell density of from 1,600 to 2,000 cpsi.

In embodiments, activated carbon honeycomb filter article can have a wall thickness of, for example, from 100 to 500 micrometers, from 100 to 250 micrometers, and from 100 to 150 micrometers, including intermediate values and ranges. One measured wall thickness for a 1600 cpsi activated carbon honeycomb filter article was about 150 micrometers.

In embodiments:

the first organic binder powder can be selected, for example, from methylcellulose, hydroxybutylcellulose, ethylcellulose, hydroxybutylmethylcellulose, hydroxyethylcellulose, hydroxymethylcellulose, hydroxypropylcellulose, hydroxypropylmethylcellulose, hydroxyethylmethylcellulose, sodium carboxylmethylcellulose, and like materials, or mixture thereof;

the second organic binder powder can be selected, for example, from sodium carboxymethycellulose, polyvinyl alcohol, chitosan, and like materials, or mixtures thereof;

the porous inorganic binder powder can be selected, for example, from a porous clay, attapulgite powder, and like materials, or combinations thereof and the extrusion aid can be selected, for example, from a surfactant, a stearate, vegetable oil, and like materials, or combinations thereof.

In embodiments, the rheological plasticizing liquid can have a viscosity of, for example, from 90 to 120 cps.

In embodiments, the honeycomb article can have a smooth, that is, no tears, cracks, blisters, or fissures, outer skin, and clear, that is, unobstructed or open channels.

In embodiments, the disclosure provides an honeycomb filter article, comprising:

an activated carbon in from 40 to 60 wt %;

a porous inorganic binder powder in from 4 to 15 wt %;

a BET surface area of from 950 m²/g to 1600 m²/g;

a cell density of from 50 to 2000 cpsi; and

a density of from 0.5 to 0.8 g/cm³.

In embodiments, the honeycomb filter article can have an iodine number of, for example, from 700 to 1,200 mg/g, which metric is an indicator of the article's excellent adsorption properties.

In embodiments, the disclosure provides a method of using the disclosed activated filter article comprising:

installing the filter article in a filter apparatus; and

passing a fluid though the installed filter article.

In embodiments, the fluid can be selected from, for example, a gas, a liquid, or a combination thereof. In embodiments, the fluid can be a volatile compound, such as an aromatic benzene or toluene, or an aliphatic hydrocarbon and like compounds, mixtures thereof, and vapors thereof.

In embodiments, the gas can be selected from, for example, a natural gas, an industrial gas, an industrial waste gas, an organic solvent vapor, ambient polluted air, an exhaust gas from a combustion engine, an exhaust gas from a combustion engine that has been treated with a catalyst, and mixtures thereof.

In embodiments, the liquid can be selected from, for example, a liquid-phase purification, a liquid-phase recovery and separation process, a natural water source, an industrial effluent stream, an organic solvent, and mixtures thereof.

In embodiments, the disclosure provides phenolic resin-free batch compositions for making activated carbon honeycomb structures. The absence of phenolic resin in the batch composition and the combination of activated carbon particles and suitable organic and inorganic binders in controlled amounts allow for successful extrusion of the batch composition and the subsequent processing of the extruded bodies into high activity, activated carbon honeycomb structures at relatively low temperatures described herein.

In embodiments, the disclosure provides for processing of the disclosed batch compositions, by for example, the following steps:

mixing into a substantially homogeneous mixture particles of: an activated carbon; a porous inorganic binder; and a first organic binder;

adding to the mixture a plasticizing liquid, prepared from a powdered second organic binder and super-addition of water, in amounts sufficient to plasticize the mixture so as to be plastically formable;

extruding the mixture at ambient temperatures, e.g., room temperature, through a suitable die to produce a honeycomb green body;

drying the resulting honeycomb green body at, e.g., 120 to 160° C., for 1 to 24 hrs, such as 2 to 10 hrs, in an air-vent oven; and

heating the dried honeycomb body at, e.g., 200 to 250° C., for 1 to 24 hrs, such as 2 to 10 hrs, in nitrogen to stabilize the honeycomb structure.

Activated carbon powders suitable for use in the disclosure are commercially available, for example, Nuchar SA-20 (1500 m²/g) and RGC (1450 m²/g) activated carbons, from Meadwestvaco, and BL activated carbon (1250 m²/g) from Calgon Corp.

The inorganic binder can be added to the batch composition to augment the binding action of the first organic binder. The inorganic binders used in the compositions can be, for example, porous clay binders, such as sepiolite powder (of the formula Mg₄Si₆O₁₅(OH)₂.6H₂O), attapulgite powder (or palygorskite; a magnesium aluminum phyllosilicate of the formula (Mg,Al)₂Si₄O₁₀(OH).4(H₂O)), and like inorganic binder materials, or combinations thereof. The amount of inorganic binder added to the batch can be, for example, from 5 to 15 wt % based on the total weight of activated carbon powder added to the batch or the total batch weight absent the water superaddition.

The powdered first organic binder can be selected, for example, from cellulose ethers, and their derivatives. The amount of first organic binder added to the batch can be, for example, from 5 to 10 wt % based on the total weight of activated carbon powder (e.g., 40 to 60 wt %) added to the batch, or the total weight of the batch composition absent the water superaddition. The first organic binder acts as a plasticizer to aid the extrusion and provides wet strength to maintain structural integrity of the extruded green shape. The first binder can be selected from the group consisting of, for example, methylcellulose, hydroxybutylcellulose, ethylcellulose, hydroxybutylmethylcellulose, hydroxyethylcellulose, hydroxymethylcellulose, hydroxypropylcellulose, hydroxypropylmethylcellulose, hydroxyethylmethylcellulose, sodium carboxylmethylcellulose, and mixtures thereof. Preferred organic binders are Methocel® products, such as Methocel (A4M), from The Dow Chemical Company.

The rheological plasticizing liquid can be prepared by dissolving a powder of a second organic binder in warm water at 60 to 70° C., for 1 to 5 hrs, such as 2 to 3 hrs, depending on the binder, to form a viscous liquid with a viscosity comparable to that of a liquid phenolic resin, such from 90 to 120 cps. The amount of second organic binder dissolved in water can be, for example, from 5 to 10 wt %. Suitable organic binders can include, for example, sodium carboxymethycellulose (CMC), polyvinyl alcohol (PVA), chitosan, and like materials, or mixtures thereof. In preparing the chitosan solution, for example, a corresponding amount such as about 5 to 10 wt % of acetic acid, based on the weight of the chitosan, was added to the water to aid in dissolving the added chitosan powder.

### EXAMPLES

The disclosure will be more fully described by the following examples. The following Examples demonstrate making, use, and analysis of the disclosed activated carbon honeycomb in accordance with the disclosed procedures.

### Example 1

An activated carbon honeycomb sample was prepared according to the disclosure and using the ingredients in Table 1. The total batch weight excluding superaddition water was 500 g. The plasticizing liquid was prepared by dissolving 5 wt % sodium carboxymethylcellulose (CMC) in warm water at 70° C. The activated carbon powder used was RGC activated carbon from MeadWestvaco having an average particle size of 20 microns and a BET surface area (SA) of 1450 m²/g. The honeycomb was prepared by mixing the batch ingredients, extruding the mixed ingredients through spaghetti die and finally extruding the spaghetti through honeycomb die (1600 cells/in²). The extruded honeycomb was dried at 140° C. in air-vented oven. The dried honeycombs were then heated at, for example, 250° C. for 2 hours in nitrogen. The activated carbon honeycomb samples were characterized for BET surface area (SA) using nitrogen adsorption, and benzene adsorption, as summarized in Example 4, and Tables 4 and 5, respectively.

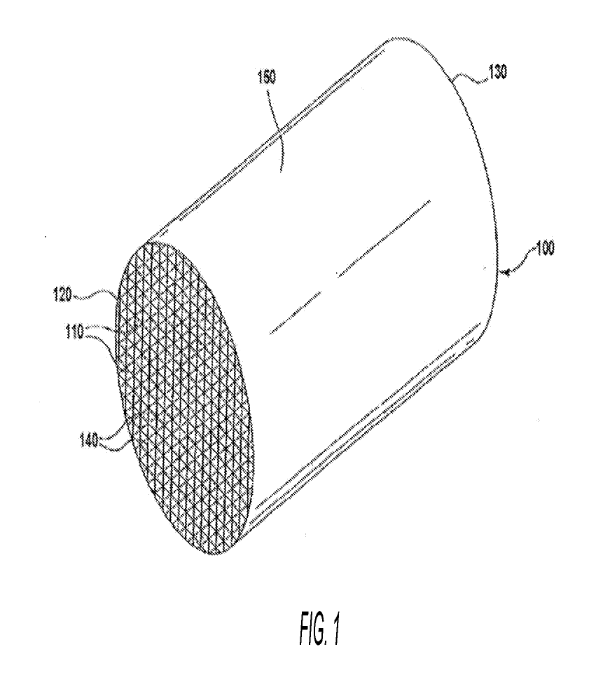

Referring to the Figures, in embodiments, the honeycomb filter articles can comprise a plurality of cell channels extending between a first and second end as shown, for example, in FIG. 1. The honeycomb structure can be suitable for use as, for example, a wall-flow gas particulate filters. A typical honeycomb flow-through substrate article 100, according to embodiments of the disclosure, is shown in FIG. 1, and can include a plurality of generally parallel cell channels 110 formed by and at least partially defined by intersecting cell walls 140 (otherwise referred to as “webs”) that extend from a first end 120 to a second end 130 and then penetrating the walls of adjacent blocked channels and exiting the filter. The channels 110 are unplugged and flow through them is straight down the channel from first end 120 to second end 130. The honeycomb article 100 can also include an optional skin 150 formed about the exterior of the honeycomb structure, and may be formed by extrusion or in later processing as an after applied skin. In embodiments, the wall thickness of each cell wall 140 for the substrate can be, for example, from about 100 to about 500 microns, from about 100 to about 150 microns, and like wall thicknesses, including intermediate values and ranges. The cell density can be, for example, from about 50 to about 2,000 cpsi, from about 500 to about 2,000 cpsi, from about 1000 to about 2,000 cpsi, from about 1250 to about 2,000 cpsi, from about 1600 to about 2,000 cells per square inch (cpsi), including intermediate values and ranges. In embodiments, the cellular honeycomb structure comprises a multiplicity of parallel cell channels 110 of generally square cross section formed into a honeycomb structure. Alternatively, other cross-sectional configurations can be used in the honeycomb structure, including, for example, rectangular, round, oblong, triangular, octagonal, hexagonal, and like geometries, or combinations thereof “Honeycomb” comprises a structure of cell walls forming longitudinally-extending cells.

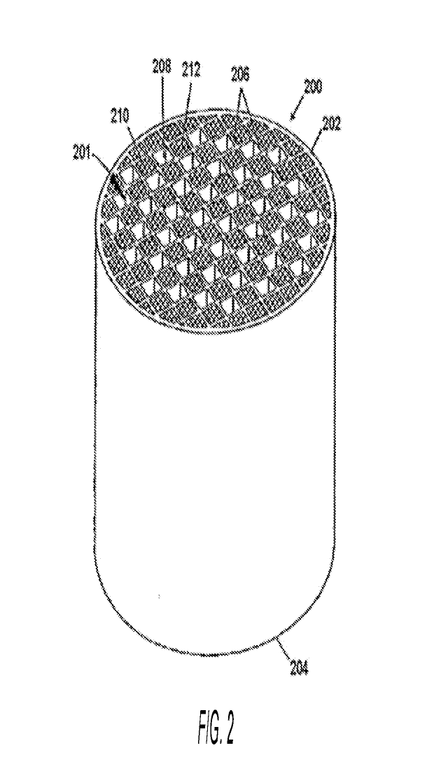

FIG. 2 illustrates an exemplary honeycomb wall flow filter 200 according to embodiments of the disclosure. The general structure includes a body 201 comprised of intersecting porous walls 206 extending from the first end 202 to the second end 204 and forming cells or channels. Certain cells are designated as inlet cells 208 and certain other cells are designated as outlet cells 210. In the filter 200, certain selected channels include plugs 212. Generally, the plugs are arranged at the ends of the channels and in some defined pattern, such as the checkerboard patterns shown. The inlet channels 208 can be plugged at the outlet end 204 and the outlet channels 210 can be plugged at the inlet end 202. Other plugging patterns can be employed and all of the outermost peripheral cells can be plugged (as shown) for additional strength. Alternatively, some of the cells can be plugged other than at the ends. In embodiments, some channels can be flow-through channels and some can be plugged providing a so-called partial filtration design.

In embodiments, the extruded and activated carbon honeycomb filter article can have, for example, a diameter of about 1 inch and a length of about 12 inches, and a honeycomb cell density of from about 50 to 2,000 cells per square inch or channel openings per square inch (cpsi). The extruded and activated carbon honeycomb filter articles can have significantly different dimensions and geometries, and the different dimensions and geometries can be achieved using appropriately structured and selected dies and extrusion equipment.

The extruded and activated honeycomb samples of the disclosure had very clear and well-defined square channels. In embodiments, immediately adjacent channel openings can be plugged or blocked (not shown) with a suitable material using known methods and materials to provide a through-wall filter article. Alternative channel geometries, such as square, rectangular, diamond, circular, and like patterns, can be selected. Alternative plugging or blocking patterns can also be selected.

TABLE 1
Batch extrusion composition for the sample prepared in Example 1.

| Ingredient | Wt % |
|---|---|
| activated carbon-RGC | 56.5 |
| inorganic binder - Sepiolite | 4.5 |
| first organic binder - Methocel A4M | 5.5 |
| extrusion aid - sodium stearate (Liga) | 1.0 |
| extrusion aid - vegetable oil | 2.5 |
| second organic binder - liquid sodium carboxymethyl cellulose | 30 |
| (CMC) 5 wt % in water | |
| water (by super-addition) | 80 |

### Example 2

The activated carbon honeycomb of this example was prepared according to the disclosure and using the ingredients in Table 2. The total batch weight excluding water was also 500 g. The plasticizing liquid was prepared by dissolving 5 wt % polyvinyl alcohol in warm water at 70° C. The activated carbon powder used was the same as in Example 1. Batching of the ingredients, extrusion of the batch composition, drying and heat treatment of the extruded body, characterization and testing of activated carbon product were carried out in the same manner as described in Examples 1 and 4.

TABLE 2
Batch extrusion composition for the sample prepared in Example 2.

| Ingredient | Wt % |
|---|---|
| activated carbon-RGC | 56.5 |
| inorganic binder - Sepiolite | 4.5 |
| first organic binder - Methocel A4M | 5.5 |
| extrusion aid - sodium stearate (Liga) | 1.0 |
| extrusion aid - vegetable oil | 2.5 |
| second organic binder - liquid polyvinyl alcohol (PVA) 5 wt % in | 30 |
| water | |
| water (by super-addition) | 75 |

### Example 3

The activated carbon honeycombs of this example were prepared according to the disclosure and using the ingredients in Table 3. The total batch weight excluding water was also 500 g. The liquid binder was prepared by dissolving 5 wt. % of chitosan in 5% v/v acetic acid solution at 60 C. The activated carbon powder used was the same as in Example 1. Batching of the ingredients, extrusion of the batch composition, drying, and heat treatment of the extruded body, and characterization and testing of extruded activated carbon product were carried out as in Examples 1 and 4.

TABLE 3
Batch extrusion composition for the sample prepared in Example 3.

| Ingredient | Wt % |
|---|---|
| activated carbon-RGC | 56.5 |
| inorganic binder - Sepiolite | 4.5 |
| first organic binder - Methocel A4M | 5.5 |
| extrusion aid - sodium stearate (Liga) | 1.0 |
| extrusion aid - vegetable oil | 2.5 |
| second organic binder - liquid chitosan 5 wt % in water | 30 |
| water (by super-addition) | 85 |

### Example 4

Honeycomb characterization and evaluation. The activated carbon honeycomb filter samples were tested for benzene adsorption by exposing the samples to benzene vapor at 40° C. for 3 hours. Additionally, a water stability test was performed by immersing the activated honeycomb filter sample in hot water at 100° C. for 6 hrs (the standard test time was 15 minutes). The activated honeycomb filter sample maintained its strength and structural integrity even after a prolonged immersion in the hot water. The BET SA and benzene uptake data are presented in Table 4 and 5, respectively. The results showed that high surface area and high benzene uptake were obtained.

TABLE 4
BET surface area of samples in Examples 1 to 3 and compared
with phenolic resin-based activated carbon honeycomb.

| | BET surface area |
|---|---|
| Sample | (m²/g) |
| Comparative Example 5 phenolic resin-based activated | 850 |
| carbon honeycomb | |
| Example 1 CMC | 1050 |
| Example 2 PVA | 1002 |
| Example 3 chitosan | 950 |

TABLE 5
Benzene uptake by Examples 1 to 3 samples and a
comparative phenolic resin based-activated carbon
honeycomb sample (exposure time was 3 hrs).

| | Benzene uptake |
|---|---|
| Sample | (wt %) |
| Comparative Example 5 phenolic resin-based activated | 35 |
| carbon honeycomb | |
| Example 1 CMC | 50 |
| Example 2 PVA | 45 |
| Example 3 chitosan | 42 |

### Comparative Example 5

Example 1 was repeated with the exception that a substantially homogeneous mixture was made by blending the ingredients (no superaddition water) in Table 6 according to conventional methods. The mixed and kneaded mass was extruded to obtain a green honeycomb body. After drying and curing the green honeycomb body at 140° C. for 2 hrs, the body was treated in a non-oxidizing atmosphere (i.e., N₂) at a temperature of from 800° C. to 900° C. for 4 hrs to produce a carbonized honeycomb body. Finally, the carbonized honeycomb body was held in an activating atmosphere (i.e., CO₂) at a temperature of from 850° C. to 950° C. for 8 to 48 hrs depending on the size of the articles. A larger honeycomb body was dried longer than a smaller honeycomb body.

TABLE 6
Batch extrusion composition for the
sample of Comparative Example 5.

| Ingredient | Wt % |
|---|---|
| charcoal | 39.8 |
| cellulose fiber (BH40) | 18 |
| Methocel A4M | 5.7 |
| sodium stearate (Liga) | 1.0 |
| vegetable oil | 2.5 |
| liquid phenolic resin | 33 |

The disclosure has been described with reference to various specific embodiments and techniques. However, it should be understood that many variations and modifications are possible while remaining within the scope of the disclosure.

1-3. (canceled)

4. A method of making an activated carbon honeycomb filter article, comprising:
extruding a batch mixture to form an extruded honeycomb body, the batch comprising:
an activated carbon powder in from 40 to 60 wt %;
a first organic binder powder in from 5 to 10 wt %;
a rheological plasticizing liquid in from 20 to 30 wt %, prepared from a second organic binder powder in from 5 to 10 wt % in water;
a porous inorganic binder powder in from 4 to 15 wt %;
an extrusion aid in from 1 to 3 wt %; and
water in from 50 to 100 wt % by superaddition,
the wt % is based on the total weight of the batch ingredients excluding water added based on superaddition;
drying the extruded honeycomb body at about 120 to 160° C. for 1 to 2 hrs; and
heat treating the dried honeycomb body at 200 to 250° C. in nitrogen for 2 to 4 hrs, to produce the activated carbon honeycomb filter article.

5. The method of claim 4 wherein the honeycomb article has a BET surface area of from 950 m²/g to 1600 m²/g.

6. The method of claim 4 wherein the extrusion is accomplished with a honeycomb die having a cell density of from 50 to 2000 cpsi, and at ambient temperature of about 15 to 30° C.

7. The method of claim 4 wherein the activated carbon honeycomb filter article has a wall thickness of from 100 to 500 micrometers.

8. The method of claim 4 wherein:
the first organic binder powder is selected from methylcellulose, hydroxybutylcellulose, ethylcellulose, hydroxybutylmethylcellulose, hydroxyethylcellulose, hydroxymethylcellulose, hydroxypropylcellulose, hydroxypropylmethylcellulose, hydroxyethylmethylcellulose, sodium carboxylmethylcellulose, or mixture thereof;
the second organic binder powder is selected from sodium carboxymethycellulose, polyvinyl alcohol, chitosan, or mixtures thereof;
the porous inorganic binder powder is selected from a porous clay, attapulgite powder, or combinations thereof; and
the extrusion aid is selected from a surfactant, a stearate, a vegetable oil, or combinations thereof.

9. The method of claim 4 wherein the rheological plasticizing liquid has a viscosity of from 90 to 120 cps.

10. The method of claim 4 wherein the extruded honeycomb article and the activated honeycomb article each has a smooth outer skin free of wrinkles and clear channels free of residual blockage.

11. The method of claim 4 wherein the extrusion is accomplished with a honeycomb die having a cell density of from 1,600 to 2,000 cpsi.

12. The method of claim 4 wherein the activated carbon honeycomb filter article has a wall thickness of from 100 to 150 micrometers.

13. A method of using the activated filter article of claim 4, comprising:
installing the filter article in a filter apparatus; and
passing a fluid though the installed filter article.

14. The method of claim 13, wherein the fluid is selected from a gas, a liquid, or a combination thereof.

15. The method of claim 14, wherein the gas is selected from a natural gas, an industrial gas, an industrial waste gas, an organic solvent vapor, ambient polluted air, an exhaust gas from a combustion engine, an exhaust gas from a combustion engine that has been treated with a catalyst, and mixtures thereof.

16. The method of claim 14, wherein the liquid is selected from a liquid-phase purification, a liquid-phase recovery and separation process, a natural water source, an industrial effluent stream, an organic solvent, and mixtures thereof.

17. The method of claim 14, wherein the fluid is a volatile organic compound.

18. The method of claim 14, wherein the fluid is benzene vapor.
